(19) United States
(12) Patent Application Publication
(10) Pub. No.: US 2016/0199573 A1
(43) Pub. Date: Jul. 14, 2016

(54) AUTONOMOUS LIQUIDS PUMP FOR TRANSPORTING A FLUID TO A LIVING BEING

(71) Applicant: MAHUSACA SPRL, Olne (BE)

(72) Inventors: Thierry Peeters, Olne (BE); François Dury, Angleur (BE); Philippe Renard, Xhoris (BE); Dimitri Gangolf, Thimister (BE)

(21) Appl. No.: 14/911,310

(22) PCT Filed: Aug. 11, 2014
(86) PCT No.: PCT/EP2014/067151

§ 371 Date: Feb. 10, 2016

(30) Foreign Application Priority Data
Aug. 13, 2013 (BE) 2013/0541

(51) Int. Cl. A61M 5/145; A61M 5/148

(52) U.S. Cl. CPC A61M 5/14586; A61M 5/148

(57) ABSTRACT

Autonomous device (10) comprising a first plate (21) having a first surface (210), a second plate (22) having a second surface (220), and an elastic mechanism (30) for bringing the first (210) and second (220) surfaces closer together and for placing a pouch (40) able to contain a fluid under pressure between said surfaces, the autonomous device (10) being configured in such a way that said first surface (210) and said second surface (220) are able to face one another, and being characterized in that said second surface (220) of the second plate (22) is convex from a viewpoint (35) situated between said first (210) and second (220) surfaces of the first (21) and second (22) plates.

### FIELD OF THE INVENTION

The invention relates to an autonomous device for transporting a fluid to a living being.

### PRIOR ART

WO2011/075104 discloses a device permitting a drug solution to be delivered to a patient. This device comprises a body having a reservoir to contain said drug solution, see for example claim 1. The device is preferably supplied to a patient in a pre-filled configuration. To fill the reservoir, it is possible to provide a device comprising an injection port as disclosed in paragraph [0063]. The device of this document comprises an integrated reservoir. The device of WO2011/075104 is not capable of receiving and compressing a bag containing a liquid to be infused.

Infusion is a procedure well known in the medical field for the slow and continuous injection of a liquid, such as a drug or a biological liquid into an organism. Such a procedure is commonly carried out, in particular, in human medicine. It is designed to compensate for losses of biological liquids, for example blood, to which an organism is subjected following, amongst other things, a surgical operation, a road accident or any circumstance having caused such losses. This is also the case, in particular, in situations of armed conflict. It is also frequently the case that the organism receiving the infusion is in a state of shock.

In principle, the infusion consists of an appropriate flow of liquid, which is contained in a flexible bag and which flows from this bag to a receiving organism via a defined circuit also called the infusion line. This flow has to be implemented at a flow rate which is as constant as possible and which is adapted to the needs of the organism receiving the infusion.

According to common practice, the bag of liquid is kept at a certain height above the organism to be supplied with the infusion, so as to ensure a flow of the liquid by gravity. This method is fairly easily applied to a hospital environment or the like, where the bag may be suspended at a constant height on a stand and accompany the patient if he/she moves. Outside, for example at the site of a road accident or natural disaster, maintaining the bag at the desired height preoccupies a member of the medical personnel who is therefore no longer available for carrying out the actual treatment. Furthermore, it is rarely possible to ensure a constant flow of liquid in these conditions.

Moreover, particularly with bags of large dimensions, this method does not always permit a sufficiently constant flow to be guaranteed, even if the bag is kept at a constant height, since the pressure of the liquid—and consequently the flow rate thereof—reduces as the bag is gradually emptied. Finally, there is a significant risk that, due to a sudden movement, the patient will tear the infusion line which connects the patient to the suspended bag.

WO94/08645 discloses an autonomous infusion device permitting these problems to be addressed. This autonomous infusion device comprises means for receiving an infusion bag and placing the infusion bag under pressure between two substantially parallel compression plates. When this device is in its normal resting position, for example placed horizontally in a cupboard, the compression plates are substantially horizontal. During the infusion, the upper plate remains fixed and the lower plate, bearing the bag, may be displaced vertically toward the upper plate by the action of an elastic mechanism. To load an infusion bag into the device, it is necessary to lift the upper plate which constitutes a foldable cover, in order to access the lower plate to position the infusion bag thereon. The closure of this foldable cover causes the loading of the elastic mechanism and the placing of the infusion bag under pressure. Thus it might be possible to describe the bag as external to the device, in the sense that it is possible to remove it from the device, for example, when it is empty.

The autonomous infusion device of WO94/08645 has certain drawbacks. It does not permit a flow of liquid from the infusion bag at a constant flow rate during the entire infusion procedure. Secondly, with this type of device it is difficult to empty the infusion bag completely.

### SUMMARY OF THE INVENTION

One of the objects of the present invention is to provide an autonomous device which is capable of receiving a bag able to contain a fluid, which is capable of compressing said bag to transport said fluid from said bag to a living being, and which has the following advantages:

a flow of said fluid from the bag containing said fluid at a more constant flow rate, and

the possibility of emptying said bag more completely.

To this end, the autonomous device according to the invention comprises:

a first plate having a first surface,

a second plate having a second surface,

said autonomous device being configured:

such that said first surface of the first plate and said second surface of the second plate are capable of opposing one another and

such that it is possible to insert said bag between said first and second surfaces of said first and second plates;

said autonomous device further comprising an elastic mechanism:

for bringing the first and second surfaces of the first and second plates together, and

for placing said bag under pressure between the first surface of the first plate and the second surface of the second plate.

The autonomous device of the invention is characterized in that said second surface of the second plate is convex from a viewpoint situated between said first and second surfaces of said first and second plates.

Said bag could be described as external in the sense that it is possible to lift it away or remove it from the autonomous device, for example when it is empty. In particular, it is possible to insert and to compress different bags, typically one after the other, using the autonomous device of the invention.

Due to the configuration of the autonomous device and to its elastic mechanism, it is possible to contain or maintain a bag between the first and second surfaces of the first and second plates. It is possible to insert said bag between the first and second surfaces of the first and second plates, for example by separating the first and second plates from one another. This is possible, for example, by removing the first plate when said plate is a cover which is foldable about a hinge. Preferably, this has the effect of unloading the elastic mechanism.

The term convex is known by the person skilled in the art. Thus, from the viewpoint between the first and second surfaces of the first and second plates, the second surface of the second plate has a convex surface, i.e. a surface which is rounded. The adjective convex is the opposite of the adjective concave, said adjective denoting a surface having a hollow shape.

The convex shape of the second surface of the second plate, from the viewpoint between the first and second surfaces of the first and second plates, makes it possible to improve the coaptation of the first and second surfaces of the first and second plates, when a bag is placed under pressure between these two surfaces, relative to the device of WO94/08645. Coaptation denotes the action or property of two surfaces to oppose one another, to be adjusted or to coincide with one another. Finally, a bag placed under pressure between the first and second surfaces of the first and second plates will spread (or extend) more effectively using the autonomous device of the invention. This permits, on the one hand, a more uniform distribution of pressure on the bag. The distribution of pressure on the bag is also more stable as the bag is gradually emptied (in other words, the pressure applied to the bag is more uniform or varies less as said bag is gradually emptied). Thus, it is possible to obtain a more uniform flow rate with the autonomous device according to the invention. On the other hand, as the bag spreads (or stretches) more effectively between the first and second surfaces of the first and second plates, the risk of the formation of folds in the bag as it is gradually emptied is reduced. More specifically, quantities of fluid to be transported may be trapped in such folds. Thus, the autonomous device of the invention makes it possible to empty a bag more completely, since the risk of the formation of folds in the bag is reduced.

Preferably, said living being is a mammal. Further preferably, said living being is a human being.

Preferably, said fluid is a liquid.

Preferably, the autonomous device is an autonomous infusion device. In this case, the fluid to be transported to a living being is generally a liquid to be supplied by infusion and the bag is generally an infusion bag.

Preferably, the autonomous device of the invention is used to permit enteral nutrition. This term is known by the person skilled in the art. Enteral nutrition is a method for substituting oral food permitting all of the necessary nutrients to be provided to an organism in order to achieve and maintain a nutritional state corresponding to the needs and characteristics of the living being to be fed. For such an enteral nutrition, a catheter is generally introduced into the digestive tract via the nose or by means of a digestive stoma. The autonomous device of the invention makes it possible to transport the nutrients present in the bag to the living being to be fed by means of such a catheter.

Preferably, the second plate is sufficiently rigid so as not to be substantially deformed when a bag is placed under pressure between the first and second surfaces of the first and second plates.

Preferably, said first surface of the first plate is concave in the absence of the bag and from a viewpoint between said first and second surfaces of the first and second plates.

This preferred embodiment preferably relates to the preferred case where the first plate is substantially rigid, i.e. to the preferred case where this first plate is subjected to little deformation (or not at all) after placing a bag under pressure by means of the elastic mechanism between the first surface of the first plate and the second surface of the second plate. In this preferred embodiment, the coaptation between the first surface of the first plate and the second surface of the second plate is improved even further since the first surface of the first plate is concave whilst the second surface of the second plate is convex from a viewpoint between these two surfaces. Thus, the flow rate of the fluid may be made even more constant and it is possible to empty the bag even more completely. Further preferably, the first surface of the first plate and the second surface of the second plate are substantially parallel (two surfaces are parallel if the line perpendicular to one is perpendicular to the other), in particular when a bag is placed under pressure between these two surfaces.

Preferably, the first plate is capable of being deformed when a bag is placed under pressure by the elastic mechanism between the first surface of the first plate and the second surface of the second plate, such that said first surface of the first plate is concave in the presence of a bag placed under pressure between the first surface of the first plate and the second surface of the second plate and from a viewpoint between said first and second surfaces of the first and second plates.

In this preferred embodiment, the first plate is capable of being deformed when a bag is placed under pressure so as to adopt a concave shape from a viewpoint between the two surfaces compressing the bag. When a bag is placed under pressure using the autonomous infusion device of WO94/08645, the foldable cover (or first plate) is deformed and adopts a bulging shape. This deformation results from the pressure exerted by the infusion bag on the foldable cover and is all the more marked when the cover (or first plate) is made of a lightweight and transparent plastics material as preferred for this type of application. It is preferred to have an autonomous infusion device which is not too heavy and to have a transparent cover (or first plate) to monitor the degree of filling of the infusion bag. More specifically, the mobile plate (or second plate) of the device of WO94/08645 is planar. Finally, the coaptation between the lower surface of the foldable cover (or first plate) and the upper surface of the mobile plate (or second plate) is poor. In any case it is less effective than that obtained by the preferred embodiment of the invention disclosed in the present paragraph. As the second surface of the second plate is convex and the first surface of the first plate is concave when a bag is placed under pressure, the coaptation between these two surfaces is improved. Finally, the flow rate of the fluid to be transported is more uniform and the risk of the formation of folds which could trap the fluid is reduced. Further preferably, the first surface of the first plate and the second surface of the second plate are substantially parallel when a bag is placed under pressure by the elastic mechanism between the first surface of the first plate and the second surface of the second plate (two surfaces are parallel if the line perpendicular to one is perpendicular to the other).

Preferably, the first surface of the first plate has a convex shape in the absence of the bag and from a viewpoint between said first and second surfaces of the first and second plates.

Preferably, the intersections of the sectional planes with the second surface of the second plate define second degree curves in the orthogonal references in these sectional planes.

Preferably, the intersections of the sectional planes with the first surface of the first plate define second degree curves in the orthogonal references in these sectional planes.

Preferably, the convexity of the second surface of the second plate (from a viewpoint between the first and second surfaces of the first and second plates) is such that this second surface of the second plate has a deflection of between 2 and 3 mm.

Preferably, the convexity of the second surface of the second plate (from a viewpoint between the first and second surfaces of the first and second plates) is such that this second surface of the second plate has a deflection of between 1 and 2 mm, and further preferably equal to 1.5 mm.

Preferably, the first plate is a cover. Further preferably, the first plate is a cover which is foldable about a hinge.

Preferably, the autonomous device further comprises a housing comprising a base.

Preferably the second plate is mobile relative to the base of the housing and said elastic mechanism is located between the base of the housing and said second plate.

Preferably, the first plate consists of a material comprising a plastics material.

Preferably, the first plate consists of a material comprising a plastics material reinforced with glass fibers.

Preferably, the second plate consists of a material comprising a plastics material.

Preferably, the second plate consists of a material comprising a plastics material reinforced with glass fibers.

The weight of the autonomous device may be reduced when the first plate and/or the second plate consist(s) of a material comprising a plastics material. Using a plastics material reinforced with glass fibers for the first and/or the second plate(s), it is possible to obtain an autonomous device which is both robust and lightweight.

Preferably, the second surface of the second plate comprises at least one lug for fixing a bag capable of containing the fluid to be transported to a living being. The presence of such a lug permits a bag pressed between the two plates to be stabilized.

Preferably, the first plate comprises at least one transparent portion. In this preferred embodiment, it is possible to observe the degree of filling of a bag inserted into the autonomous device, and in a simple manner.

Preferably, the elastic mechanism is directly coupled to the second plate.

Preferably, the autonomous device further comprises monitoring means to control the degree of emptying of a bag.

Preferably, a projection of the second surface of the second plate over a plane has substantially the shape of a rectangle.

Preferably, the first surface of the first plate is a lower surface of the first plate.

Preferably, the second surface of the second plate is an upper surface of the second plate.

### BRIEF DESCRIPTION OF THE FIGURES

These features and other features of the invention will be clarified in the detailed description of preferred embodiments of the invention, reference being made to the drawings of the figures, in which:

FIG. 1 shows schematically a section of an autonomous device according to a first preferred embodiment without a bag and with a bag;

FIG. 2 shows schematically a section of an autonomous device according to a further preferred embodiment without a bag and with a bag;

FIG. 3 illustrates the determination of a deflection characterizing the convex deformation of the second surface of the second plate;

FIG. 4 shows a perspective view of the second surface of the second plate for a preferred embodiment of the invention as well as a section of such a second surface along a vertical plane;

FIG. 5 shows a plan view of the second surface of the second plate according to a further preferred embodiment;

FIG. 6 shows a section of a further preferred embodiment of the device of the invention without a bag and with a bag;

FIG. 7 shows a section of a further preferred embodiment of the device of the invention without a bag and with a bag.

The drawings of the figures are not to scale. Generally, the same elements are denoted by the same reference numerals in the figures. The presence of reference numerals in the drawings should not be considered as limiting, including when these numerals are indicated in the claims.

### DETAILED DESCRIPTION OF PARTICULAR EMBODIMENTS

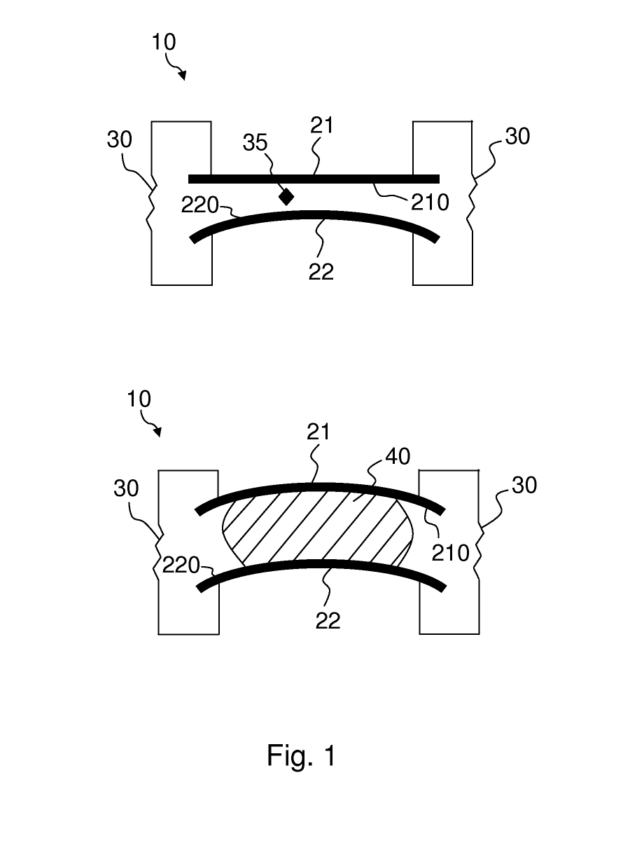

FIG. 1 shows schematically the autonomous device 10 according to a first preferred embodiment. The upper part of FIG. 1 shows the autonomous device 10 without the bag 40. The autonomous device 10 comprises a first plate 21 having a first surface 210. It also comprises a second plate 22 having a second surface 220. An elastic mechanism 30 makes it possible to bring the first surface 210 of the first plate 21 toward the second surface 220 of the second plate 22. As may be seen in the lower part of FIG. 1, this elastic mechanism 30 also permits a bag 40 to be placed under pressure between the first surface 210 of the first plate 21 and the second surface 220 of the second plate 22. This bag 40 enables a fluid to be contained, said fluid being intended for transportation to a living being. As may be seen in FIG. 1, the first plate 21 is preferably capable of being deformed when a bag 40 is placed under pressure by the elastic mechanism 30 between the first surface 210 of the first plate 21 and the second surface 220 of the second plate 22. The autonomous device 10 is configured such that the first surface 210 of the first plate 21 and the second surface 220 of the second plate 22 may be placed opposite one another. The second surface 220 of the second plate 22 is convex from the viewpoint 35 between the first 210 and the second 220 surfaces of the first 21 and the second 22 plates. Thus, from the viewpoint 35, the second surface 220 of the second plate has the shape of a boss, it is convex; it is not concave, it does not have a hollow shape. Once the bag 40 is placed under pressure between the two surfaces 210 and 220, the first plate 21 is deformed in the preferred embodiment illustrated in FIG. 1 due to the pressure exerted by the bag 40 and said bag is pressed between two surfaces 210 and 220 which have improved coaptation relative to the device disclosed in WO94/08645. This is possible due to the convex shape of the second surface 220 of the second plate 22 from the viewpoint 35.

In the preferred embodiment which is illustrated in FIG. 1, the first surface 210 of the first plate 21 is substantially planar in the absence of the bag 40. In a further preferred embodiment (not shown), the first surface 210 of the first plate 21 is convex in the absence of the bag 40 and from the viewpoint 35 between the two surfaces 210 and 220. In this same preferred embodiment, the first plate 21 is capable of being deformed when a bag 40 is placed under pressure by the elastic mechanism 30 between the first surface 210 of the first plate 21 and the second surface 220 of the second plate 22 such that the first surface 210 of the first plate 21 is concave when a bag 40 is placed under pressure between the two surfaces 210 and 220. Thus, when a bag 40 is placed under pressure between the two surfaces 210 and 220, there is also an effective coaptation between these two surfaces 210 and 220.

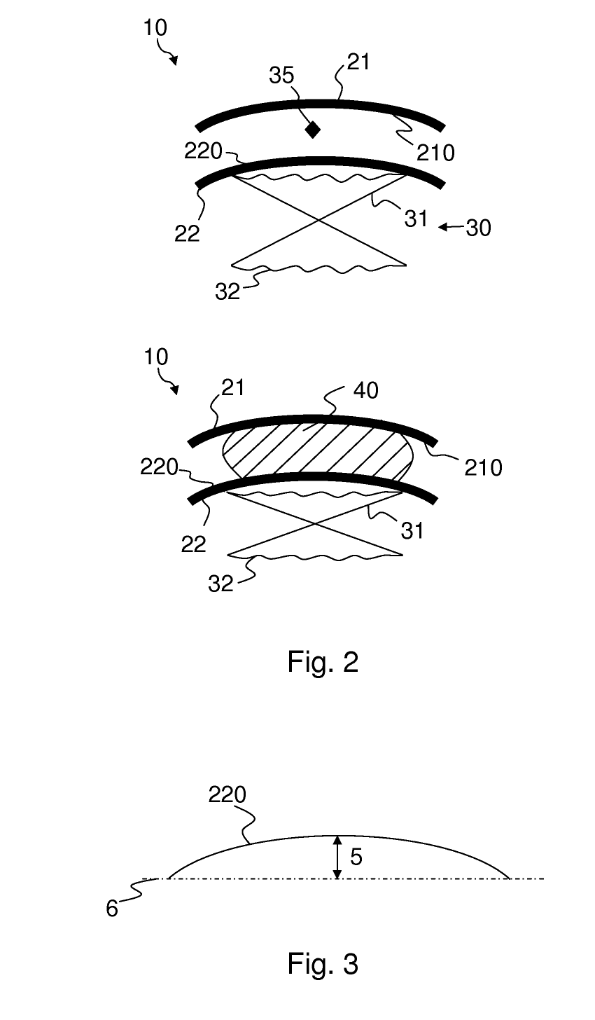

FIG. 2 shows a further preferred embodiment. As may be seen in this figure, the first surface 210 of the first plate 21 is preferably concave in the absence of the bag 40 and from a viewpoint 35 between the first 210 and second 220 surfaces of the first 21 and second 22 plates. The second surface 220 of the second plate 22 is convex from the same viewpoint 35. Thus, ultimately the distribution of pressure on a bag 40 placed under pressure between the two surfaces 210 and 220 is more uniform and it is possible to empty the bag 40, limiting the risk of the formation of folds in which the fluid to be transported may be retained. The elastic mechanism 30 shown in FIG. 1 could be used with the preferred embodiment shown in FIG. 2 and vice-versa. Thus, the elastic mechanism 30 of FIG. 2 which comprises two intersecting levers 31 joined by springs 32 in traction could be used with the preferred embodiment which is shown in FIG. 1. In the embodiment shown in FIG. 2, a mechanism (not shown) makes it possible to maintain the first plate 21 in a certain position so that it exerts a pressure on the bag 40 which in turn transmits a pressure onto the second surface 220 of the second plate 22 permitting the springs 32 to be tensioned, said springs tending to push the second plate 22 toward the first plate 21. This may be a loading mechanism, for example, which permits the first plate 21 to be locked in a specific position.

Preferably, the convexity of the second surface 220 of the second plate 22 is such that this second surface 220 has a deflection 5 of between 2 and 3 mm for all the preferred embodiments of the invention. However, this deflection 5 may be greater. Thus, for example, this deflection 5 may be 3.5, 4, 4.5 or 5 mm. The deflection 5 is defined as the maximum value of the deflection of the second surface 220 of the second plate 22 relative to a planar surface 6. FIG. 3 shows how this deflection 5 is determined. Preferably, for the preferred embodiment shown in FIG. 2, the concavity of the first surface 210 of the first plate 21 is such that this first surface 210 has a deflection of between 2 and 3 mm. However, this deflection of the first surface 210 may be greater. Thus for example, this deflection may be 3.5, 4, 4.5 or 5 mm.

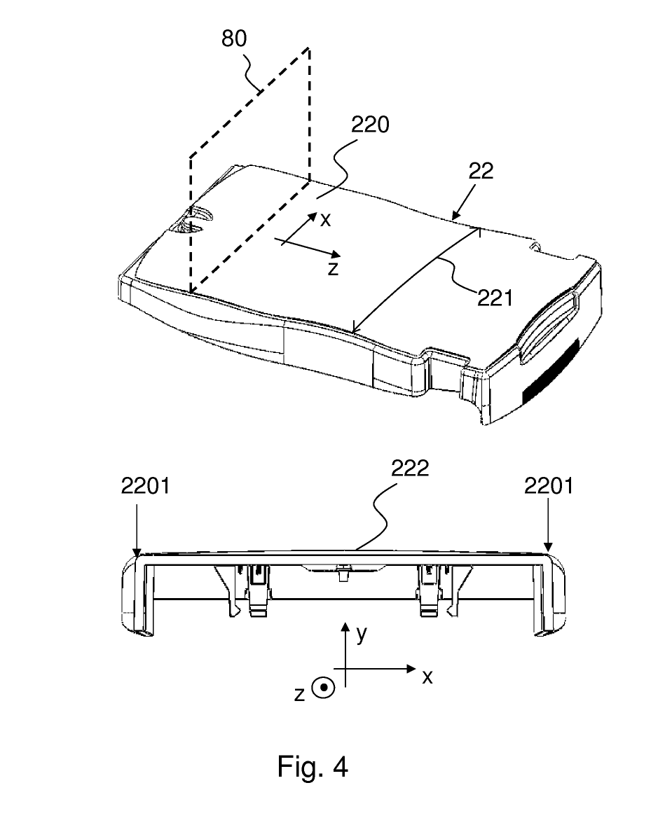

In a further preferred embodiment, the second surface 220 of the second plate 22 which is convex from a viewpoint 35 situated between the two surfaces 210 and 220 has a shape such that the intersections of the sectional planes 80 with this second surface 220 define second degree curves in the orthogonal references in these sectional planes 80. This preferred version is illustrated in FIG. 4. The top of FIG. 4 shows a perspective view of the second surface 220 of the second plate 22 for this preferred embodiment of the invention in addition to a vertical sectional plane 80. The bottom of FIG. 4 shows the intersection of this vertical sectional plane 80 with the second surface 220 of the second plate 22. This intersection of the sectional plane with the second surface 220 of the second plate 22 defines a curve 222. Preferably, this curve 222 is a second degree curve in the x-y orthogonal reference shown at the bottom of FIG. 4. In order to quantify the convex deformation of the second surface 220, an origin of the axis x is selected which is located at half of the average width 221 of the second surface 220 of the second plate 22. Thus, the distance of the curvature between x=0 and each of the two ends 2201 of the curve 222 shown at the bottom of FIG. 4 is the same (assuming that the two ends 2201 are separated by the average width 221). Preferably, if these two ends 2201 of the curve 222 are located at y=0, the point of the curve 222 at the coordinate x=0 has a y-coordinate of between 0.5 and 3 mm (this means that the deflection 5 is between 0.5 and 3 mm). Further preferably if these two ends 2201 of the curve 222 are located at y=0, the point of the curve 222 where x=0 has a y-coordinate of between 1 and 2.5 mm (this means that the deflection 5 is between 1 and 2.5 mm). Preferably, the convex deformation of the second surface 220 of the second plate 22 is not constant (or uniform) along the z-axis, i.e. along the length of the second plate.

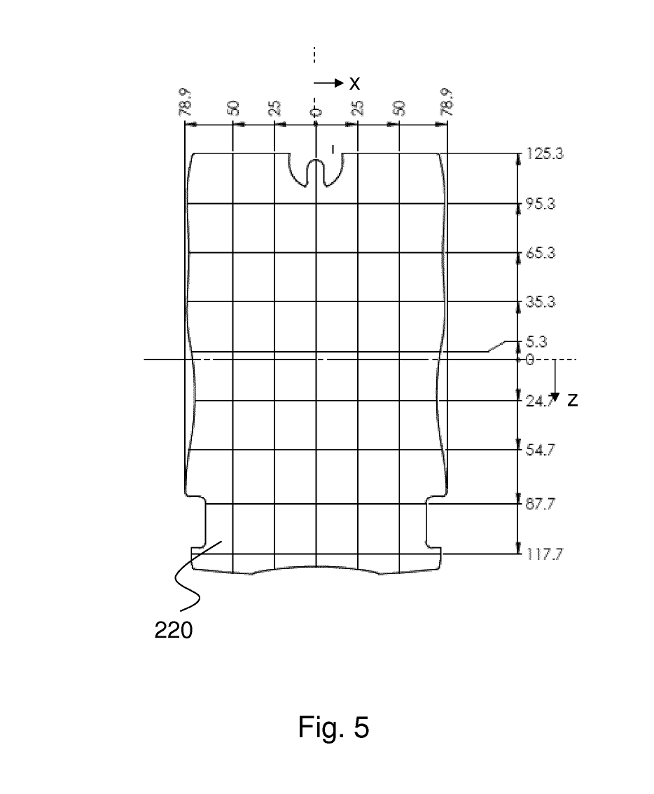

In a further preferred embodiment, the second surface 220 of the second plate 22 has a shape such that the intersections of the sectional planes 80 with this second surface 220 define second degree curves in orthogonal references x-y located in these sectional planes 80, these second degree curves having the following equations with different z-coordinates shown in FIG. 5 (an example of the sectional plane 80 is shown at the top of FIG. 4). FIG. 5 is a plan view of the second surface 220 of the second plate 22 where it is possible to see the origin of the x-axis and z-axis (the coordinates along these axes are expressed in mm). The y-axis is in turn perpendicular to the plane of FIG. 5 (and thus perpendicular to the x-axis and z-axis).

Second Degree Equations in the x-y Reference Defining the Intersections of the Second Surface 220 with the Sectional Planes 80 Located at Different z-Coordinates for the Preferred Embodiment Illustrated in FIG. 5:

where z=117.7 mm: y=0.0003x²−5*10⁻⁵x+2.5407;

where z=87.7 mm: y=−0.0003x²−6*10⁻⁵x+2.4888;

where z=54.7 mm: y=−0.0003x²−6*10⁻⁵x+2.4555;

where z=24.7 mm: y=0.0002x²−4*10⁻⁵x+2.417;

where z=5.3 mm: y=0.0002x²−4*10⁻⁵x+2.3152;

where z=−35.3 mm: y=−0.0002x²−4*10⁻⁵x+2.1111;

where z=−65.3 mm: y=−0.0001x²−4*10⁻⁵x+1.7707;

where z=95.3 mm: y=7*10⁻⁵x²−10⁻⁵x+1.2648;

where z=−125.3 mm: y=−2*10⁻⁵x²−7*10⁻⁶x+0.5742.

In these difference equations, the x- and y-coordinates are expressed in mm. By calculating the difference in the y-coordinates for x=0 and x=78.9 mm, it is possible to deduce the estimations of the different deflections 5 for the different z-coordinates. Thus, where z=117.7 mm, it is deduced that the deflection 5 is approximately 0.669 mm (replacing x in the first equation by 78.9 mm).

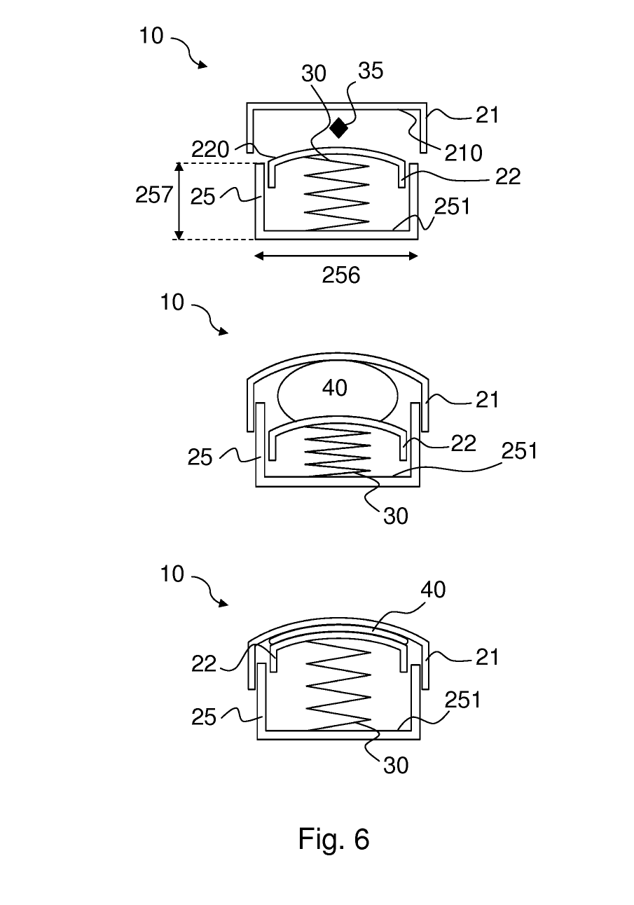

FIG. 6 shows a section of a further preferred embodiment of the autonomous device 10 of the invention without a bag and with a bag 40. In this preferred embodiment, the first plate 21 is capable of being deformed when a bag 40 is placed under pressure by the elastic mechanism 30 between the first surface 210 of the first plate 21 and the second surface 220 of the second plate 22 such that said first surface 210 of the first plate 21 is concave in the presence of a bag 40 placed under pressure between the two surfaces 210 and 220 from a viewpoint 35 between these two surfaces 210 and 220. In this preferred embodiment, the autonomous device 10 further comprises a housing 25 which comprises a base 251. The first plate 21 is a foldable cover coupled to the housing 25. The second plate 22 is mobile relative to the base 251 of the housing 25 and the elastic mechanism 30 is located between the base 251 of the housing 25 and the second plate 22. The upper illustration of FIG. 6 shows the autonomous device 10 without the bag 40 whilst the central and lower illustrations of FIG. 6 show the autonomous device 10 when a bag 40 is placed under pressure between the first surface 210 of the first plate 21 and the second surface 220 of the second plate 22. The central illustration of FIG. 6 corresponds to the case where the bag 40 is substantially filled, whilst the lower illustration of FIG. 6 shows the configuration where the bag 40 is substantially empty. A loading system (not shown) permits the first plate 21 (foldable cover) to be held in a fixed position relative to the housing 25 when it is closed onto the top thereof (see the central and lower illustrations of FIG. 6). Due to such a fixed position of the first plate 21, a bag 40 may be placed under pressure between the first 210 and second 220 surfaces of the first 21 and second 22 plates by tensioning the elastic mechanism 30. Preferably, the autonomous device 10 comprises means for locking the second plate 22 to hold said second plate in a loading position, permitting the bag 40 to be put in position. The loading position of the mobile second plate 22 is the position in which it is located when the elastic mechanism 30 exerts a maximum restoring force to bring the second surface 220 of the second plate 22 toward the first surface 210 of the first plate 21. For the preferred embodiment of FIG. 6, positioned as illustrated in the same FIG. 6, this loading position corresponds to a lower position of the second plate 22.

In this preferred variant, the autonomous device 10 also comprises means for unblocking said second plate 22 from its loading position, for the purpose of permitting the movement thereof toward its resting position. The resting position of the second mobile plate 22 is the position in which it is located when the elastic mechanism 30 exerts a minimum restoring force to bring the second surface 220 of the second plate 22 toward the first surface 210 of the first plate 21. The resting position generally corresponds to the position in which a bag 40 placed between the first 210 and second 220 surfaces is substantially empty. When an elastic mechanism 30 as illustrated in FIG. 2 is used, the loading position corresponds to the situation where the springs 32 are tensioned to the maximum, whilst the resting position corresponds to the situation where the springs 32 are tensioned to the minimum.

Preferably, the housing 25 has a length of 150 to 300 mm (measured perpendicular to the plane of FIG. 6). Preferably, the sections of the housing 25 perpendicular to this length define U-shaped sections as illustrated in FIG. 6. Preferably, the width 256 of this U-shape is between 100 and 200 mm. Preferably, the height 257 of this U-shape is between 40 and 80 mm. These preferred dimensions permit the device 10 to receive commonly used infusion bags 40.

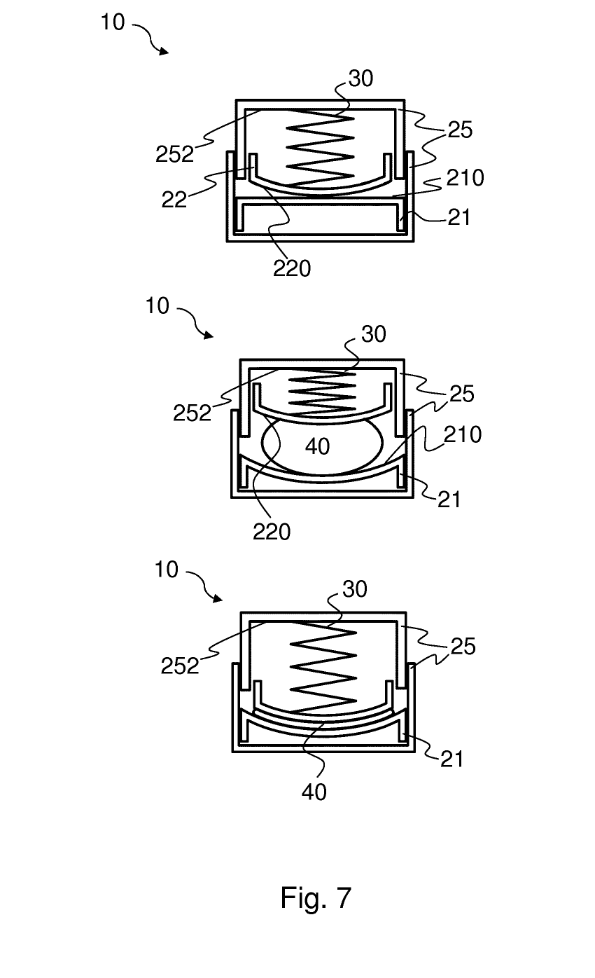

FIG. 7 shows a further preferred embodiment of the autonomous device 10. As may be seen in this figure, the elastic mechanism 30 is located between the upper wall 252 of the housing 25 and the second plate 22. A bag 40 may be arranged on the first surface 210 of the first plate 21. In this preferred embodiment, the first plate 21 is capable of sliding in the manner of a drawer. This permits the bag 40 to be inserted into the autonomous device 10. In this preferred embodiment which is illustrated in FIG. 7, the first plate 21 is subjected to a concave deformation when a bag 40 is placed under pressure between the first 210 and the second 220 surfaces.

Preferably, the second surface 220 of the second plate 22 comprises at least one lug for fixing a bag 40. Further preferably, the second surface 220 of the second plate 22 comprises two such lugs.

Preferably, the first plate 21 substantially consists of a plastics material. Examples of such plastics materials are: polyethylene, polyamides. Further preferably, the first plate 21 substantially consists of a reinforced plastics material. Even more preferably, the first plate 21 substantially consists of a plastics material reinforced with glass fibers. In a further preferred embodiment, the first plate 21 substantially consists of a transparent plastics material. This transparent plastics material may comprise a transparent plastics material reinforced with glass fibers.

Preferably, the second plate 22 substantially consists of a plastics material. Further preferably, the second plate 22 substantially consists of a reinforced plastics material. Even more preferably, the second plate substantially consists of a plastics material reinforced with glass fibers.

Preferably, the first plate 21 comprises at least one transparent portion. This makes it possible to display the filling rate of the bag 40.

Preferably, the autonomous device 10 comprises monitoring means for controlling the degree of emptying of the bag 40. Examples of such monitoring means which may be used individually or combined with one another are:

a system comprising an encoder wheel and an opto-electronic sensor detecting the variation in height of either the first 21 or second 22 plates:

an audio and/or visual system for signaling the end of the infusion operation:

an audio and/or visual system for signaling any malfunction occurring during the infusion: loss of pressure, blockage of the infusion line and the like;

flow meter, preferably a mass flow meter;

means for displaying the flow rate;

alarm system in the event of a variation in the flow rate beyond predetermined limits.

The autonomous device 10 of the invention does not need to be in a particular position to operate. By placing a bag 40 under pressure between the first 210 and second 220 surfaces of the first 21 and second 22 plates, the autonomous device 10 is able to operate in any position. Preferably, the elastic mechanism 30 comprises a pre-loaded and self-lubricating mechanism consisting of small articulated connecting rods and pre-loaded spring elements so as to increase the compressive force which tends to bring the first 210 and second 220 surfaces of the first 21 and second 22 plates together.

The present invention has been described with reference to specific embodiments which are purely illustrative and should not be considered as limiting. Generally, the present invention is not limited to the examples illustrated and/or described above. The use of the verbs: “comprise”, “include”, “consist” or any other variant and the conjugations thereof does not in any way exclude the presence of elements which are different from those mentioned above. The use of the indefinite article: “a” or the definite article: “the” to introduce an element does not exclude the presence of a plurality of these elements. The reference numerals in the claims do not limit the scope thereof.

In summary, the invention may also be described as follows. Autonomous device 10 comprising a first plate 21 having a first surface 210, a second plate 22 having a second surface 220, an elastic mechanism 30 to bring the first 210 and second 220 surfaces together and to place a bag 40 under pressure therebetween, the autonomous device 10 being configured such that said first surface 210 and said second surface 220 are capable of opposing one another and being characterized in that said second surface 220 of the second plate 22 is convex from a viewpoint 35 situated between said first 210 and second 220 surfaces of said first 21 and second 22 plates.

1. An autonomous device which is capable of receiving a bag, which may contain a fluid, and which is capable of compressing said bag in order to transport said fluid from said bag to a living being, said autonomous device comprising:
a first plate having a first surface,
a second plate having a second surface,
said autonomous device being configured:
such that said first surface of the first plate and said second surface of the second plate are capable of opposing one another, and
such that it is possible to insert said bag between said first and second surfaces of said first and second plates;
said autonomous device further comprising an elastic mechanism:
for bringing the first and second surfaces of the first and second plates closer together, and
for placing said bag under pressure between the first surface of the first plate and the second surface of the second plate;
wherein said second surface of the second plate is convex from a viewpoint situated between said first and second surfaces of said first and second plates.

2. The autonomous device as claimed in claim 1, wherein said first surface of the first plate is concave in the absence of the bag and from a viewpoint between said first and second surfaces of the first and second plates.

3. The autonomous device as claimed in claim 1, wherein the first plate is capable of being deformed when a bag is placed under pressure by the elastic mechanism between the first surface of the first plate and the second surface of the second plate, such that said first surface of the first plate is concave in the presence of a bag placed under pressure between the first surface of the first plate and the second surface of the second plate, and from a viewpoint between said first and second surfaces of the first and second plates.

4. The autonomous device as claimed in claim 3, wherein the first surface of the first plate has a convex shape in the absence of the bag and from a viewpoint between said first and second surfaces of the first and second plates.

5. The autonomous device as claimed in claim 1, wherein the intersections of the sectional planes with the second surface of the second plate define second degree curves in the orthogonal references in these sectional planes.

6. The autonomous device as claimed in claim 1, wherein the intersections of the sectional planes with the first surface of the first plate define second degree curves in the orthogonal references in these sectional planes.

7. The autonomous device as claimed in claim 1, wherein said convexity of the second surface of the second plate is such that said second surface of the second plate has a deflection of between 2 and 3 mm.

8. The autonomous device as claimed in claim 1, wherein the convexity of the second surface of the second plate is such that this second surface of the second plate has a deflection of between 1 and 2 mm.

9. The autonomous device as claimed claim 1, wherein the first plate is a cover.

10. The autonomous device as claimed in claim 1, wherein it further comprises a housing comprising a base.

11. The autonomous device as claimed in claim 10, wherein the second plate is mobile relative to the base of the housing and in that said elastic mechanism (30) is located between the base of the housing and said second plate.

12. The autonomous device as claimed in claim 1, wherein the first plate includes a plastics material.

13. The autonomous device as claimed in claim 12, wherein the first plate includes a plastics material reinforced with glass fibers.

14. The autonomous device as claimed in claim 1, wherein the second plate includes a plastics material.

15. The autonomous device as claimed in claim 1, wherein the second surface of the second plate comprises a lug for fixing said bag.

16. The autonomous device as claimed in claim 2, wherein the first plate is capable of being deformed when a bag is placed under pressure by the elastic mechanism between the first surface of the first plate and the second surface of the second plate, such that said first surface of the first plate is concave in the presence of a bag placed under pressure between the first surface of the first plate and the second surface of the second plate, and from a viewpoint between said first and second surfaces of the first and second plates.

17. The autonomous device as claimed in claim 16, wherein the first surface of the first plate has a convex shape in the absence of the bag and from a viewpoint between said first and second surfaces of the first and second plates.

18. The autonomous device as claimed in claim 8, wherein the deflection is 1.5 mm.
